(19) United States
(12) Patent Application Publication
(10) Pub. No.: US 2026/0360510 A1
(43) Pub. Date: Nov. 12, 2026

(54) Long-term Intelligent Memory for Behavioral Intervention and Change Using Reasoning Associative Graphs

(71) Applicant: GAIA AG, Hamburg (DE)

(72) Inventors: Olaf Nackenhorst, Jesteburg (DE); Bernhard Wellhöfer, Hamburg (DE); Mario Weiss, Hamburg (DE)

(21) Appl. No.: 19/203,299

(22) Filed: May 9, 2025

(51) Int. Cl. G16H 20/70; G06F 16/3329; G06F 16/334; G06F 40/284

(52) U.S. Cl. CPC G16H 20/70; G06F 16/33295; G06F 16/3347; G06F 40/284

(57) ABSTRACT

A digital therapist is implemented in a computer that has a conversation history; a first heuristic for establishing clinical significance of tokens in the conversation history; a triplet extractor generating from the conversation history a graph of nodes connected by relationships; a second heuristic for scoring strength of clinically-significant relationships among nodes; and a graph-traversing engine for identifying interventions that may be effective for given symptoms.

### TECHNICAL FIELD

Embodiments of the disclosure relate to conversation engines implemented in computers, for example, engines that generate therapeutic dialogues with one or more patients for behavioral interventions.

### BACKGROUND

Presently, generative artificial intelligence systems ("GenAI") are prevalent. Such systems use statistical guessing to produce a most likely correct response to a prompt. They lack rigor in the algorithms that generate their responses, they are prone to mistaken guesses called "hallucinations,” and there is not a high likelihood that a particular response will be "correct" in a useful sense of that term. Accordingly, GenAI is not appropriate for result-sensitive tasks such as providing professional service.

An example of a results-sensitive task is Cognitive Behavioral Therapy (“CBT”), sometimes referred to as “talk therapy.” In the CBT treatment modality, a patient or client converses with a trained professional to enhance the patient’s functioning with any of a range of psychological disorders including depression, PTSD, etc.

Treatment sessions are traditionally held in person in a comfortable setting between therapist and patient in order to promote communication and engagement. The treatment seeks to help patients become more self-aware and recognize factors that influence their emotional well-being, and also to encourage supportive behaviors and activities so that patients can reach and maintain their own emotional balance.

It is generally known that a series of regular CBT sessions is necessary to reinforce treatment initiatives until patients themselves become aware of improvements in their emotional state.

Ubiquitous Internet connectivity and the rise of mobile computing devices have made it possible for CBT to be consumed by patients without visiting a therapists’ office. The patient’s own environment and schedule can be more easily accommodated to not only leverage their personal comfort, but also to expand delivery of CBT services.

Computerized therapy systems are known, including systems for providing CBT, but these systems are only “intelligent” in the sense that they have the ability to answer a limited number of questions or provide a limited amount of information. Additionally, such systems have been only text-based. They either cannot accept inputs other than text, or they only provide replies in text, or both. So, the efficacy of existing computerized CBT systems is limited at least because the full range of the therapists’ observations and experience cannot be used for treatment.

Further, current generation computerized therapy systems, including systems which may be operated as “chat bots,” have trouble with logic and reasoning because they are fundamentally statistical guessing machines that produce the most “likely” response to a prompt. For example, existing systems may have trouble counting how many "r" letters are in the word “strawberry.” The reasons for these troubles are fairly simple and also are fundamental to how these systems work (i.e., existing systems may represent “strawberry” as two or three tokens and count how many tokens have an “r”). When handling signals of intense emotional valence rather than spelling words, the potential for over-simplification by an unsupervised computerized therapy system could be counter-productive or even dangerous.

It would be desirable to provide a computerized CBT system that could generate responses with a relatively high likelihood of being "correct" as in usable for a results-sensitive purpose. Examples of results-sensitive purposes include dialogs that provide an end-user with therapeutic or advisory results, e.g., talk therapy, legal counseling, business advisement.

According to aspects of the disclosure, a digital therapist is implemented in a computer that has a conversation history; a first heuristic for establishing clinical significance of tokens in the conversation history; a triplet extractor generating from the conversation history a graph of nodes connected by relationships; a second heuristic for scoring strength of clinically-significant relationships among nodes; and a graph-traversing engine for identifying interventions that may be effective for given symptoms.

In some aspects, the nodes include life events, emotions, symptoms, behaviors, skills, and interventions. The relationships may include triggered by, associated with, recommended for, helped with, and leads to.

In some aspects, the computer also has a structured summary of the graph. Such a structured summary may include an interconnection analysis of the graph. For example, the interconnection analysis may include notable patterns. The structured summary may include a clinical analysis of the graph. Such a clinical analysis may include an identification of management gaps in an intervention plan. The structured summary may include actionable recommendations for interventions.

According to aspects of the disclosure, a digital therapist is implemented in a computer that has a memory storing a conversation history with a patient; a triplet extractor transforming the conversation history into at least one graph of clinically-significant nodes and relationships; a graph database storing the one or more graphs; a vector embedder transforming the conversation history into a plurality of multidimensional vectors; the same or another memory also storing a set of most recent messages; a query generator generating a query based on the most recent messages as context; a vector database generated by combining the query, one of the graphs, and one or more of the vectors; a composer generating a message based on the vector database; and the message output to the patient.

In some aspects, the computer also may include a pathmatcher generating anchors in the graph database based on combinations of graphs with queries. In some aspects, the computer also may include IRCoT (Interleaving Retrieval with Chain-of-Thought) expansion of a query generated by the query generator, prior to combining the query with the graph. In some aspects, the computer also may include a multi-hop graph ranking prior to combining the query with the graph.

According to aspects of the disclosure, a digital therapist is implemented in a computer that is configured to implement a method, which includes receiving a conversation comprising tokens of text; identifying tokens of clinical significance, wherein the tokens of clinical significance include nodes and relationships; building a graph of clinically-significant nodes connected by clinically-significant relationships; and traversing the graph for identifying interventions that may be effective for given symptoms.

In some examples, the nodes include life events, emotions, symptoms, behaviors, skills, and interventions. In some examples, the relationships include triggered by, associated with, recommended for, helped with, and leads to.

In some examples, the method also includes a structured summary of the graph. The structured summary may include an interconnection analysis of the graph. The interconnection analysis may include notable patterns. The structured summary may include a clinical analysis of the graph. The clinical analysis may include an identification of management gaps in an intervention plan. The structured summary may include actionable recommendations for interventions.

Other features and aspects of the present teachings will become apparent from the following detailed description, taken in conjunction with the accompanying drawings, which illustrate by way of example the features in accordance with embodiments of the present teachings. The summary is not intended to limit the scope of the present teachings

### BRIEF DESCRIPTION OF THE DRAWINGS

FIG. 1 depicts a patient graph 100, according to an aspect of the disclosure, in which “nodes” including events 102, emotions 104, symptoms 106, behaviors 108, interventions 110, and outcomes 112 are linked by “edges” or relationships including “happened to”, “helps with”, and “triggered by”.

FIG. 2 depicts an architecture for a digital therapist 200 that incorporates the patient graph 100 to drive a composer 202 that generates a next response to a conversation history 10.

### DETAILED DESCRIPTION

Aspects of the disclosure make use of long-term intelligent memory for behavioral intervention and change using reasoning associative graphs. “Long-term intelligent memory” and “reasoning associative graphs” will be further discussed.

A persistent intelligent memory system can enable an LLM-based system to approximate “reasoning”. It is currently believed that, in the human brain, long-term intelligent memory is achieved by the hippocampus. Neuroscientists believe that the hippocampus binds memory to time and place. Inspired by hippocampal memory indexing theory (human memory), embodiments of the disclosure mimic the functions of human neocortex and hippocampus with natural language processing, knowledge graphing, and a PersonalizedPageRank algorithm. Based on this mimicking, embodiments of the disclosure outperform other knowledge retrieval devices - such as vector-based semantic search engines, classical question-answering (QA) systems, or retrieval-augmented generation (RAG) pipelines - by up to 20%, while being ~10× faster/cheaper.

One application for embodiments of the disclosure would be digital therapy. Long-term intelligent memory could be applied not only to facts, but in a multilevel approach that connects events, emotions, behaviour, symptoms, interventions, and outcomes. An aspect of intelligent memory is restriction (“pruning”) of relationships among memory nodes, so as to condense and rationalize systemic knowledge to focus on therapy relevant things and relationships. Neural pruning is an important process in the development of human intelligence from infancy into adulthood; in one theory of neuroscience, the human brain makes sense of things by discarding inapt neural connections. According to an aspect of the disclosure, discrete data (nodes) can be linked in memory only by expert-defined (pre-programmed) relationships (edges).

Embodiments of the disclosure provide memory for each patient with strict isolation. The memory graph for a patient tends to be sparse. This supports dynamic development of the patient construct, not represented by hierarchical traversal of the graph. Rather than pure storage and retrieval of memory, embodiments of the disclosure impose relational reasoning on the memorized data.

In a more detailed and advanced approach, embodiments of the disclosure emulate the supposed functions of the cingulate gyrus (emotional and cognitive connections), the thalamus (filtering information for relevance), the hypothalamus (behavioral regulation), the amygdala (emotional learning), and the hippocampus (memory storage and retrieval). The thalamic function in particular is developed in Reasoning Associative Graphs (RAGs). These are weighted graphs based on temporal decay and domain knowledge of clinically significant relationships among memory nodes. The RAGs are constructed using domain-knowledge pathfinding, cause-and-effect relationships, and retrieval augmented generation.

By combining node synonyms with measurements of importance across the domain-specific relationships, embodiments of the disclosure can emulate reasoning to answer questions such as:
1. Which intervention helped the patient in the past with anxiety? (*:Intervention) –[HELPED_WITH]→ (Anxiety:Emotion) (PMR) –[HELPED_WITH]→ (Anxiety) (Mediation) -[HELPED_WITH]→ (Anxiety)
2. Which events triggered the anxiety of the Patient? (*:LifeEvent) –[triggered]→ (Anxiety:Emotion) (Job Loss) –[TRIGGERED]→ (Anxiety) (Harsh performance review) -[TRIGGERED]→ (Anxiety)

Embodiments of the disclosure implement a graph ranking system (e.g., in imitation of the amygdala, which imparts emotional valence to remembered events). In the graph ranking system, each edge of the graph has a relation score on a [0…1] scale, a frequency score (0.5 + (0.1)*additional occurrence, max 1), a therapeutic score based on domain knowledge (nodes, relations, triplets), and an importance score (LLM based for therapeutic and patient relevance). The graph ranking system imparts temporal decay to rankings (exponential with 1 year half-life), as a model of “forgetting”.

Additionally, embodiments of the disclosure implement subgraph ranking, which calculates a weighted average of 3 scores times decay. Weights are user defined; sum is
1. (Job Loss) –[TRIGGERED]→ (Anxiety)
(PMR) –[HELPED_WITH]→ (Anxiety)
(Meditation) –[HELPED_WITH]→ (Anxiety)

In certain aspects, embodiments of the disclosure implement multi-hop retrieval or “deep thinking.” For example, which events triggered the anxiety of the Patient?

Since yoga helped the patient with insomnia and panic attacks (which are symptoms of anxiety), it also could help the patient also with anxiety.
(Job Loss) –[TRIGGERED]→ (Anxiety)
(Job Loss) –[LEADS_TO]→ (Insomnia)
(Yoga) –[HELPED_WITH]→ (Insomnia)
(Yoga) –[HELPED_WITH]→ (Panic attacks)

Embodiments of the disclosure include a patient analyzer that converts a patient memory graph to mermaid code, then extracts full or N-hops around a seed node to generate a universe of relationships among nodes. Embodiments of the disclosure feed the mermaid code to an LLM that has expert knowledge as context for the input. The LLM analyzes the graph and produces a structured summary with interconnection analysis based on the expert knowledge. This supports deeper clinical analysis.

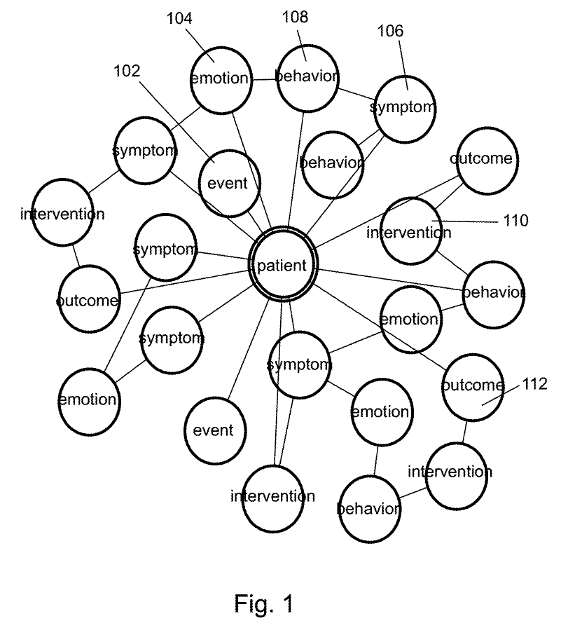

As described more generally above, FIG. 1 depicts a patient graph 100, according to an aspect of the disclosure, in which “nodes” including events 102, emotions 104, symptoms 106, behaviors 108, interventions 110, and outcomes 112 are linked by “edges” or relationships including “happened to,” “helps with,” and “triggered by.”

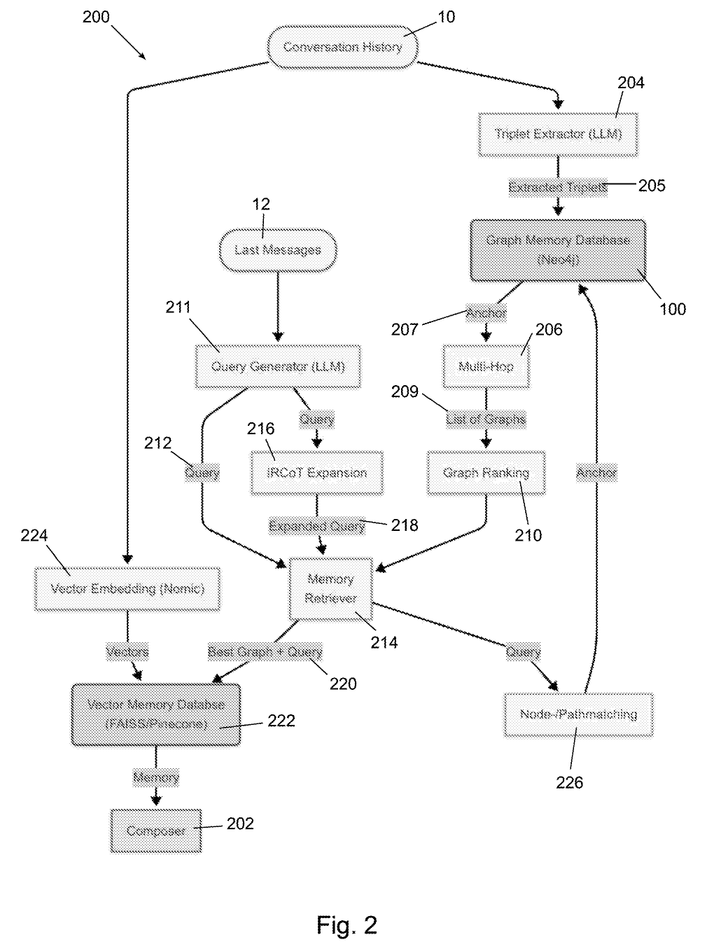

Meanwhile, FIG. 2 depicts an architecture for a digital therapist 200 that incorporates the patient graph 100 to drive a composer 202 that generates a next response to a conversation history 10. As an initial matter, the digital therapist 200 continually produces and updates the patient graph 100 using a triplet extractor 204 that extracts triplets 205 from the conversation history 10; the graph 100 comprises the triplets 205, e.g., (Job Loss) –[TRIGGERED]→ (Anxiety)

as discussed above. At 206, the digital therapist selects anchors 207 and performs multi-hop reasoning from each anchor through the patient graph 100 to produce a list of graphs 209. At 210, the digital therapist generates rankings of the graph relationships discussed with reference to FIG. 1. Meanwhile, the digital therapist also includes a query generator 211, which works with a set of last or recent messages 12 to generate queries 212 that drive a memory retriever 214. Optionally, an IRCoT expansion 216 may augment the queries 212 to produce expanded queries 218, which can enhance the functioning of the memory retriever. Combining the queries 212 or enhanced queries 218 with the graph rankings 210, the memory retriever 214 provides a best graph and query 220 representing the most semantically and structurally relevant combination of user intent and domain knowledge.

To arrive at this best combination, the memory retriever first intelligently fuses the generated or expanded queries with the ranked graph structures in order to retrieve relevant prior information from long-term memory. This process goes beyond traditional keyword search by leveraging context-aware retrieval techniques that incorporate both the semantic embedding of the query and the structural features of the graph, such as node centrality and path salience. The retrieval process typically employs vector-based similarity search technologies - for example, using FAISS or Pinecone - to locate prior interactions or knowledge fragments that most closely match the user’s current context.

From the resulting pool of candidate matches, the system then identifies the optimal graph-query pair (220). This selection is based on a combination of factors, including semantic relevance to the current dialogue, structural coherence within the patient graph, and the expected effectiveness of memory retrieval. The selected pair functions as a focused access key to the subsequent vector memory retrieval step, ensuring that the response generation process is grounded in the most contextually and structurally appropriate knowledge available.

This best graph and query 220 are subsequently passed to a vector memory database 222 (using the FAISS/Pinecone protocol), which relies on vector embedding 224 on the conversation history 10 to feed the composer 202. Additionally, at 226 the digital therapist implements node- and path-matching to update the patient graph 100 by emphasizing selected anchors 207.

What is claimed is:

1. A digital therapist, implemented in a computer comprising:
a conversation history;
a first heuristic for establishing clinical significance of tokens in the conversation history;
a triplet extractor generating from the conversation history a graph of nodes connected by relationships;
a second heuristic for scoring strength of clinically-significant relationships among nodes;
a graph-traversing engine for identifying interventions that may be effective for given symptoms.

2. The digital therapist of claim 1, wherein the nodes include life events, emotions, symptoms, behaviors, skills, and interventions.

3. The digital therapist of claim 1, wherein the relationships include triggered by, associated with, recommended for, helped with, and leads to.

4. The digital therapist of claim 1, further comprising a structured summary of the graph.

5. The digital therapist of claim 4, wherein the structured summary includes an interconnection analysis of the graph.

6. The digital therapist of claim 5, wherein the interconnection analysis includes notable patterns.

7. The digital therapist of claim 5, wherein the structured summary includes a clinical analysis of the graph.

8. The digital therapist of claim 7, wherein the clinical analysis includes an identification of management gaps in an intervention plan.

9. The digital therapist of claim 7, wherein the structured summary includes actionable recommendations for interventions.

10. A digital therapist, implemented in a computer comprising:
a memory storing a conversation history with a patient;
a triplet extractor transforming the conversation history into at least one graph of clinically-significant nodes and relationships;
a graph database storing the one or more graphs;
a vector embedder transforming the conversation history into a plurality of multidimensional vectors;
the same or another memory also storing a set of most recent messages;
a query generator generating a query based on the most recent messages as context;
a vector database generated by combining the query, one of the graphs, and one or more of the vectors;
a composer generating a message based on the vector database; and
the message output to the patient.

11. The digital therapist of claim 10, further comprising a pathmatcher generating anchors in the graph database based on combinations of graphs with queries.

12. The digital therapist of claim 10, further comprising IRCoT expansion of a query generated by the query generator, prior to combining the query with the graph.

13. The digital therapist of claim 10, further comprising a multi-hop graph ranking prior to combining the query with the graph.

14. A digital therapist, implemented in a computer configured to implement a method comprising:
receiving a conversation comprising tokens of text;
identifying tokens of clinical significance, wherein the tokens of clinical significance include nodes and relationships;
building a graph of clinically-significant nodes connected by clinically-significant relationships; and
traversing the graph for identifying interventions that may be effective for given symptoms.

15. The digital therapist of claim 14, wherein the nodes include life events, emotions, symptoms, behaviors, skills, and interventions.

16. The digital therapist of claim 14, wherein the relationships include triggered by, associated with, recommended for, helped with, and leads to.

17. The digital therapist of claim 14, the method further comprising a structured summary of the graph.

18. The digital therapist of claim 17, wherein the structured summary includes an interconnection analysis of the graph.

19. The digital therapist of claim 18, wherein the interconnection analysis includes notable patterns.

20. The digital therapist of claim 18, wherein the structured summary includes a clinical analysis of the graph.

21. The digital therapist of claim 20, wherein the clinical analysis includes an identification of management gaps in an intervention plan.

22. The digital therapist of claim 18, wherein the structured summary includes actionable recommendations for interventions.
